(19) United States
(12) Patent Application Publication
(10) Pub. No.: US 2013/0231719 A1
(43) Pub. Date: Sep. 5, 2013

(54) ARRANGEMENT FOR ADJUSTING THE UVB TO UVA RATIO OF ARTIFICIAL UV LIGHT

(71) Applicants: Attila Soltesz-Nagy, Miskolc (HU); Laszlo Polyak, Miskolc (HU)

(72) Inventors: Attila Soltesz-Nagy, Miskolc (HU); Laszlo Polyak, Miskolc (HU)

(73) Assignee: Attila SOLTESZ-NAGY, Miskolc (HU)

(21) Appl. No.: 13/884,865

(22) PCT Filed: Oct. 12, 2011
(86) PCT No.: PCT/HU2011/000097

§ 371 Date: May 10, 2013

(30) Foreign Application Priority Data
Nov. 12, 2010 (HU) P1000611
Jun. 1, 2011 (HU) P1100288

(51) Int. Cl. A61N 5/06

(52) U.S. Cl. CPC A61N 5/0614
USPC 607/88

(57) ABSTRACT

The invention relates to an arrangement for optimally adjusting the UVB to UVA ratio of artificial UV light, particularly for solar cosmetic purposes, comprising at least one light source emitting in both the UVB and UVA wavelength ranges, where the major part of spectral power of the light source falls in the range between 321-400 nm, preferably between 321-365 nm, and where at least one single-layer UV filter is arranged between the light source and the irradiated surface, The invention is essentially characterised by that the transmittance of the at least one UV filter at the wavelength of 305 nm or below is lower than 5% and at the wavelength of 320 nm or above is greater than or equal to 59%, with the transmission of the filter at the wavelength of 313 nm being in the range of 12-59%. The UV filter according to the invention comes near the UVB to UVA ratio that is optimal from the aspect of tanning efficiency by providing that the transmittance curve (3) has a steeper upward slope than the transmittance curves of known filters.

### TECHNICAL FIELD

The present invention relates to an arrangement for optimally adjusting the UVB to UVA ratio of artificial UV light, particularly for solar cosmetic purposes, comprising at least one light source emitting in both the UVB and UVA wavelength ranges, with the major part of spectral power of the light source falling in the range between 321-400 nm, preferably between 321-365 nm, and with at least one single-layer UV filter being arranged between the light source and the irradiated surface. The arrangement according to the invention may be utilised advantageously in fields where artificial UV radiation has beneficial effects but there are constraints on the UVB to UVA ratio.

### BACKGROUND ART

It is well known that tanning is a result of the reaction of the human body to ultraviolet radiation. Due especially to the depletion of the ozone layer protecting the Earth's surface from UV radiation, outdoor sunbathing has become more and more dangerous in recent decades. Exaggerated sunbathing leads to sunburns, manifested as erythema and blistering of the skin. Recent research has shown that serious skin diseases, such as melanoma, usually do not result from regular sunbathing but rather from repeated (and in some cases serious) sunburns. At the same time, tanning also has its benefits that include the strengthening of the immune system, the prevention of osteoporosis, increased vitality, and increased vitamin D production. The positive effects of sunlight on people's mood by relieving depression should not be neglected either.

Tanning devices utilising artificial light sources have recently become increasingly popular and more and more widely used. Such devices are for instance sun tanning beds and sun tanning booths that expose the user to UV radiation in order to provide a tanning effect.

A great advantage of artificial tanning devices is that irradiation time and intensity can be controlled and thereby exposure can be adapted to needs and requirements set by the different skin types. High-intensity irradiation, which could cause burns quickly, can be avoided to provide a healthy tanning.

UV light sources emit UVC, UVB and UVA radiation that, if mixed in a right proportion, may produce the feel and effect of natural sunlight. Because, among its other effect, it disrupts the DNA of germs, UVC radiation may be used for disinfection or water purification. Due to these harmful effects the human body must not be exposed to UVC radiation, and therefore this wavelength range has to be filtered out in artificial sun tanning and phototherapy devices. UVB radiation contributes to the tanning process and highly stimulates vitamin D production. However, in greater amounts it may cause melanoma through DNA damage, and may also cause eye damage. UVA has beneficial effects on the human body as it fosters bone formation and tanning, but in greater doses it may damage collagen fibres, cause premature skin aging, and may also disrupt vitamin A in the skin.

It is therefore of utmost importance that the intensity of UV light, and in particular the UVB to UVA ratio is controlled such that the health of the user is preserved while at the same time vitamin D production and healthy tanning are provided. The primary effects of UVB are that it oxidises (and thus turns to a darker colour) the previously generated melanin particles, and that it provides vitamin D production up to a wavelength of 318 nm. UVA radiation stimulates melanogenesis in the melanocytes of the skin. Excess UVB radiation may cause burns on the irradiated surface, but the lack of UVB hinders melanin oxidation and vitamin D production. The tendency of UV radiation to cause sunburn decreases with increasing wavelength, and thus the amount of UVB radiation emitted by current sun tanning devices has been further reduced. In sunbeds, sunbooths and phototherapy devices it is necessary to provide lighting with a predetermined UVB to UVA ratio to achieve the most beneficial biological effects and to avoid harms.

There are therefore two things essentially required for tanning. One essential component is UVB radiation, which is primarily responsible for continuous pigmentation provided by melanin oxidation. The most efficient UVB wavelength range is around ca. 300 nm, the stimulating effect on vitamin D production being the strongest here. In the higher region of the UVB band, at around 318 nm, however, the vitamin D generating effect almost completely disappears. The other key factor is instant pigmentation, that is the formation of melanosomes and melanogenesis, which are helped largely by UVA radiation, with the most effective wavelength range lying between 320 nm and 365 nm.

For burn-free tanning it is expedient to apply filters that “tame” UVB radiation by reducing erythema-inducing components to safe levels, while leaving other spectral components virtually intact.

In recent years the fluorescent tubes applied in artificial sun tanning devices have become more and more powerful, with both the percentage of emitted UVB and the emitted power of the machines increasing in order to provide shorter tanning times for clients. Market competition and client demand for shorter and cheaper tanning sessions both reinforced this trend. The erythema action time (burn time) and the time required for pigmentation were essentially the same, with vitamin D production, pigmentation and melanin oxidation all having carried out at the same time. In many cases, burns had occurred before pigmentation could have started.

In order to protect consumers from the harmful effects of exposure to artificial sunlight, the newest European Union regulation aims at reducing the UV, especially UVB irradiance, of the skin during sunbed sessions to reduce the risk of burns. According to the regulation the UV emission of sunbeds has been maximised. The limit for emitted UV power, weighted by the erythema effectiveness curve, is 0.3 W/m² that corresponds to the irradiance measured at the equator at local noon. Thereby, burn time has significantly increased at the cost of reduced melanogenesis and D vitamin production, while the relative tanning efficiency and also the economic efficiency of tanning devices having been also reduced.

The so-called erythema effectiveness curve specifies the share of each wavelength region in inducing erythema, sunburn, and blistering of the skin. The higher the erythema factor for a given wavelength interval, the shorter time is needed for the given UV spectral region to induce sunburn. In the low UV regions the erythema factor is 1, while above 298 nm it approaches 0 as wavelength increases, and in the higher UVA region it almost reaches 0. The erythema effectiveness curve in itself clearly shows those regions that are responsible for the most drastic erythema effect. From around 298-300 nm the curve slopes down significantly, and continues to slope down until 320 nm but not as rapidly. From the aspect of tanning efficiency higher UVA regions, such as the region around 380 nm, are not as effective as the 321 nm wavelength.

An ideal filter would transmit less than 5% of incident light at 305 nm, while at 313 nm it would transmit 12-59% of the light, and at 320 would substantially reach 100% transmittance. When all other transmittance criteria are taken into account, it is impossible to reach the ideal 100% transmittance at 320 nm. Therefore, considering the above cited transmittance requirements at 305 nm and 313 nm, it can be argued that the filter should have a transmittance of at least 59% at 320 nm, which can be feasible utilising special glass or plastic materials.

Taking all aspects into account, the highest efficiency may be obtained for artificial tanning devices and phototherapy devices, such as sunbeds, sunbooths, and tanning lamps, by applying phosphors with a resonance peak at a wavelength of maximum 390 nm, but preferably below 365 nm, and providing that the majority of the UVA spectral power of the light source lies in the wavelength range of 321-400 nm, and preferably in the range of 321-365. The application of a light source conforming to these requirements together with a suitable filter provides that the skin surface to be tanned may be irradiated with optimum efficiency because thereby the skin surface is exposed to light having optimum spectral distribution for tanning effect, and at the same time the erythema limit values may be reached with lower electric power. Skin burns are avoided, while vitamin D production, melanin oxidation and pigmentation occur normally, and the difference between burn time and the time needed for pigmentation may be chosen arbitrarily such that for any given skin type the erythema-inducing exposure time is longer than the time necessary for inducing pigmentation.

In addition to conforming to the power limits specified in the regulations, the optimal adjustment of the UVB to UVA ratio is also important for healthy tanning and for the economical operation of the tanning device. On the one hand, the adjustability of the UVB to UVA ratio allows that the power of the device may be chosen corresponding to skin type. On the other hand, in addition to the controlled filtering of UVB, efficiency is increased by applying UV lighting having the highest possible UVA content.

The prior art contains a number of solutions for adjusting the UVB to UVA ratio of UV light.

One group of the known solutions applies UVB and UVA filters placed between the light source and the irradiated surface, the desired UVB/UVA ratio being set by mechanically adjusting the position of the filters. Such a solution is disclosed in the patent DE 3,927,301. Patent specification DE 3,422,605 A1 relates to an apparatus providing whole-body irradiation. The apparatus comprises fluorescent tubes arranged at a small distance beside one another that produce UVA rays while UVC radiation is filtered out completely and UVB rays are filtered out partially. In the region near the head of the person to be irradiated miniature fluorescent tubes are arranged between the longer, parallelly arranged tubes extending along the whole length of the device. The spectral distribution of the light emitted by the miniature tubes is the same as that of the light of the larger tubes.

According to another group of known solutions either the glass of the light source is made from a material absorbing a portion of UVB radiation, or the glass of the light source is provided with UVB absorbing coating. U.S. Pat. No. 5 350 972 relates to UV absorbing lamp glass. The glass may be applied for producing fluorescent lamp envelopes that absorb UVB radiation at wavelengths between 280-320 nm due to its cerium oxide and iron oxide content. U.S. Pat. No. 4,615,989 relates to a CuO-bearing, alkali- and alkaline-earth rich phosphate glass. The glass produced according to the specification has sharply increasing UV transmittance in the wavelength region of 310-340 nm. The document EP 615,277 discloses a lamp having varying composition along its length, resulting in a higher UVB emission at one of the lamp's ends. The document U.S. Pat. No. 7,163,904 describes a special borosilicate glass having a UV filtering layer. The document U.S. Pat. No. 7,598,191 relates to a gas discharge lamp made with an UV-absorbing borosilicate glass, and a method for producing the same. The document U.S. Pat. No. 7,375,043 relates to an UV-absorbing glass. The document US 2009/0206720 describes low-pressure discharge lamps having indium tin oxide coating.

According to a further group of known solutions multiple layers of filter foil are placed between the light source and the target. The document U.S. Pat. No. 7,172,294 describes a multilayer film filter for display panels or projection type displays. The material of the filter layers is chosen such that they have a step difference in transmittance at longer wavelengths (400 nm).

The patent EP 0,267,655 relates to a sunbathing filter with incomplete UVB absorption. The filter according to the specification substantially transmits all UVA rays in the 320-400 nm range, while it almost completely absorbs UVB radiation in the range of 290-310 nm, and it absorbs at least 80% of UVB radiation between 310 nm and 320 nm. The disadvantage of the invention is that it absorbs too much of the UVB radiation necessary for tanning. A further disadvantage is that filtering factors cannot be exactly assigned to wavelength values.

The closest prior art to the present invention is described in the document WO 2004/090589 A1, which relates to a sun protection film for protecting the human skin, hair, or eyes from the harmful effects of natural or artificial sunlight. The multilayer-structure film has at least one filter layer that is transparent to radiation over a predetermined, or predeterminable wavelength range, and is combined with at least one transparent and/or colourless supporting layer. To provide optimum transmittance values, one or more layers of the filter have perforations arranged in different patterns. Perforations are realised as circular holes having a diameter of 0.3-10 mm. The invention described in the patent specification has a number of disadvantages. First, the applied multiple filter layers deteriorate filtering efficiency, and second, because the filter layers and the supporting layers have different material composition, individual layers undergo mechanical and chemical changes, as well as colour changes to a different extent, and also degrade at a different speed.

### DISCLOSURE OF INVENTION

The objective of the present invention is to adjust the UVB to UVA ratio of light emitted by artificial light sources such that the highest possible efficiency is achieved utilising the lowest power possible. A further objective is to utilise those wavelength regions of UVA radiation that provide the highest tanning effect. UVA radiation above 320 nm should be let through the filter to the largest possible extent such that it reaches the surface to be irradiated, while the UVB region causing skin burn in a short time should be cut off from the UVB wavelength region. A still further objective of the invention is that, besides setting the optimal UVB to UVA ratio, the skin characteristics of the tanning client are also taken into account to provide sufficient vitamin D production and melanin oxidation.

The invention is based on the recognition that from the aspect of tanning efficiency the operation of an arrangement is optimal if the selected UV light source emits radiation in the widest possible spectral region and with the highest possible intensity in the 321-400 nm, but preferably in the 321-365 nm UVA wavelength region such that meanwhile the filtered UVB radiation is kept under a predetermined value. To fulfil these requirements such a light source is needed where the resonance peak of the applied phosphors is under 390 nm, and preferably under 365 nm, and the major part of the UVA spectral power lays between 321-365 nm. Thereby the highest possible tanning efficiency is achieved utilising a given UV light source, with vitamin D production being optimal, while the difference between burn time and pigmentation time is kept at the value corresponding to the given skin type. Such a filter is needed that has the shortest possible transition wavelength range between the minimum and maximum transmittance values, the range having a width of preferably 15-20 nm, more preferably 5-15 nm.

In the present specification the term “minimum transmittance” refers to a transmittance less than 5% at 305 nm, while the term “maximum transmittance” refers to a transmittance greater than or equal to 59% at 320 nm. Transmittance between 310-320 nm is generally at least 20.1%, and is preferably between 45-55%.

Sunbeds and sunbooths presently apply low- and high-pressure UV light sources. The UV wavelength region of these light sources inducing tanning lays in the UVA/UVB regions. UVC emission by these lights sources is almost entirely prohibited. According to US regulations the wavelength range of UVC radiation is 0-260 nm, the ranges of UVB and UVA radiation, being 261-320 nm and 321-400 nm, respectively. According to European regulations the wavelength range of UVC radiation is 0-280 nm, the ranges of UVB and UVA radiation, being 281-315 nm and 316-400 nm, respectively.

To set the desired UVB to UVA ratio tanning lamp manufacturers usually apply a phosphor layer on the inside surface of the glass envelope of a low-pressure germicidal light source. Radiation emitted at the excited Hg lines, more particularly the two main Hg lines around 185 nm and 253.7 nm reaches the phosphor layer and, in different spectral distribution depending on the parameters of the applied phosphor, gets converted to radiation in the UVB-UVA and visible wavelength regions. The phosphors applied in tanning lamps may be excited by light at the wavelengths of 185, 253.7, 302, and 313 nm.

It should be emphasised here that in all tanning lamps the majority of UVB radiation is provided by the Hg lines located around 313 nm. The other characteristic Hg line is located around 365 nm. In low-pressure tanning lamps a more even spectral distribution between the two Hg peaks is provided by the exciting the applied phosphor. More particularly, in the so-called EU 0.3 fluorescent tubes mainly the region above 360 nm is saturated utilising phosphor, with the 313 nm region at the lower part of the UV range is almost completely removed utilising a glass envelope to decrease the erythema effect. The above effect is produced in high-pressure tanning lamps by adding metal halides to the lamp, and thus, due to the high pressure and temperature, and to the presence of Hg, UV power appears also in the regions between the characteristic Hg lines. Because the glass envelope of these lamps transmits a small amount of UVC radiation, they can only be used with UV filters. The filters currently applied are made from a glass material that significantly decreases shorter-wavelength UVA efficiency.

According to the current state of the art, in case of both low- and high-pressure lamps the transition range from 0 to maximum transmittance of the glass envelope of lamps is 40-80 nm wide. This implies that in order to be able to utilise a phosphor having a resonance peak at 350 nm, for instance BaSi2O5:Pb, with the best possible efficiency, such a glass material is required that reaches its maximum transmittance at 320 nm. In that case, however, UV light should be transmitted beginning at as low a wavelength as 280 nm, which would mean that the erythema-inducing UVC and UVB regions would also be let through in such amounts that the client would get burnt before melanogenesis could be started or melanin oxidation could occur. Because of that, either the lowest transmitted wavelength is shifted upwards or a wider-band transmittance curve is applied by the manufacturers so that UVB emission can be kept under control. This, however, results in a reduction of useful UVA radiation, because lower UVA wavelengths having high tanning efficiency cannot reach the skin surface. Another option is to utilise such phosphors, for instance SrB4O7:EU or BaSO4:EU, that have their resonance peaks at higher wavelengths, for instance at 370 nm or 375 nm. The excitation minimum of these phosphors is around 345 nm. Utilising the above described glass materials the highest possible efficiency of these phosphors can be achieved while keeping UVB radiation under control, but tanning efficiency is a little lower than in case of the above mentioned phosphor type (BaSi2O5:Pb) because of the lack of lower-wavelength UVA radiation, and thus higher electric power is required to reach the tanning capacity of the BaSi2O5:Pb phosphor.

The UV filter according to the present invention comes near the UVB to UVA ratio that is optimal from the aspect of tanning efficiency by providing that the transmittance curve has a steeper upward slope than the transmittance curves of known filters.

The present invention fulfils its objective by providing an arrangement having the features detailed in claim 1. Further advantageous embodiments are described in the dependent claims.

The filter is made from polyester, preferably from poly(ethylene terephthalate) (PET) or poly(ethylene naphthalate) (PEN) or from a material in the group of the derivatives thereof. In a further preferred embodiment the filter is made from cellulose-based material. According to the invention the material of the filter is polyethylene (PE), or polypropylene (PP), or the copolymers thereof, or acrylic, but in this case the filter has to be coated so as to provide sufficient filter lifetime and to achieve the objectives of the invention. It is expedient to apply an indium tin oxide (ITO) layer as coating, where the thickness of the ITO coating should be between 0.1-5 μm, preferably between 3-4 μm.

By changing the thickness of the filter the intensity of the 313 nm Hg line, that is, the filter's transmittance may be adjusted, and thereby the desired UVB to UVA ratio may be set. In case the thickness of the filter is increased, the transmittance curve is shifted towards the higher wavelengths. According to the invention the thickness of the filter is lower than 100 μm, and is preferably between 5-40 μm.

In a preferred embodiment of the invention the filter is mounted on and/or secured to the plastic sheet surfaces that separate the irradiated space from the light source of the sun tanning device. The filter may be disposed on a load-bearing surface if so required, provided that the thickness of the surface is above 2000 μm. Despite the very small thickness of the filter it is not necessary to apply a supporting layer. In case of low-pressure tanning lamps the filter may be disposed directly on the surface of the lamp.

In addition to adjusting the thickness of the filter, the optimal UVB to UVA ratio may also be achieved by removing material from the filter, for instance by perforating the filter surface. Thereby the UVB/UVA ratio of the light hitting the irradiated surface may be adjusted without changing the chemical composition of the filter material. The intensity of radiation reaching the irradiated surface through a filter having through holes in the filter surface can be calculated as follows.

At a wavelength λ a device has an intensity (per unit surface area) E_{λ}. The transmittance of the filter at the wavelength A is F_{λ}, a value between 0 and 1. The combined intensity at λ is E_{Fλ}=E_{λ}×F_{λ}. For a perforated filter the overall area of the filter (to which unfiltered radiation is directed with almost even intensity distribution) is A, and the non-perforated area through which the radiation hits the irradiated surface is A_{F}, The ratio of the non-perforated and the total area is T=A_{F}/A (0 . . . 1). The combined transmittance of the perforated filter at a wavelength λ is F_{Pλ=}1−T+F_{λ}×T. The combined filtered intensity at a the wavelength λ is

E_{Pλ}=E_{λ}×F_{Pλ}, that is E_{Pλ}E_{λ}(1−T+F_{λ}×T).

Perforations may be disposed across the entire surface of the filter or may only be disposed across individual sections of the filter. The shape and size of the perforation may change across the surface of the filter. It may therefore be advantageous to divide the irradiated surface into zones having different-intensity incoming radiation. The same device may thereby be applied for instance for tanning the face with higher intensity than other body parts. By choosing the size, shape and distribution of the perforations the intensity of the filtering may be optimally adjusted. It is also possible to counteract the weakening of fluorescent tubes by changing the thickness of the filter.

In case of low-pressure tanning lamps, where the filter is placed directly on the lamp surface, zones with different intensity may be created. By reducing the perforations and/or layer thickness of the filter the intensity reduction of the tanning lamp may be compensated.

Another important parameter of the filter is the HAZE factor measuring opacity. With increasing transparency (increasing the HAZE factor) the transmittance curve becomes less steep, that is, it is shifted towards higher wavelengths. According to a preferred embodiment the HAZE factor value of the filter is between 0-5, and preferably between 0-2.

Optimal parameters for every potential application may be determined by the proper selection of the material, thickness, and HAZE factor of the filter according to the invention. According to a preferred application, by applying multiple filters having identical or different parameters different erythema values or different UVB to UVB ratios may be achieved in a single arrangement.

### BRIEF DESCRIPTION OF DRAWINGS

The invention is explained in further detail below referring to the preferred embodiments and the accompanying figures, where

FIG. 1 shows a curve series illustrating the biological effect of UV radiation,

FIG. 2 is the transmittance curve of a prior art glass filter, and

FIG. 3 shows the transmittance curves of three UV filters according to the invention.

### BEST MODE OF CARRYING OUT THE INVENTION

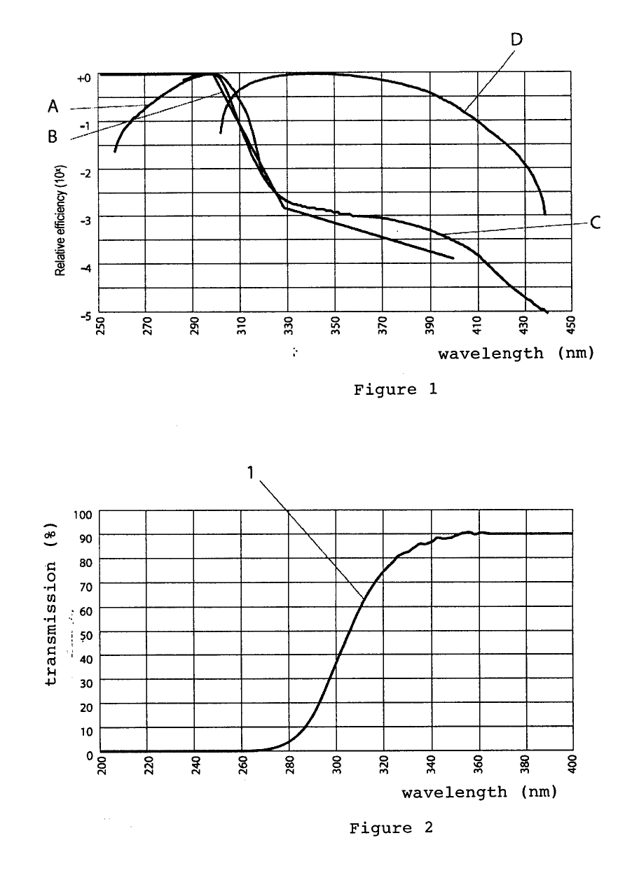

FIG. 1 illustrates the biological effect of UV radiation, where the curves show the relative efficiency as a function of wavelength. Curve A indicates vitamin D production, curve B illustrates erythema effect, curve C shows melanin oxidation, and curve D shows melanosome formation.

FIG. 2 illustrates the characteristics of a conventional glass filter known from the prior art. On the vertical axis of the diagram transmittance is displayed in percents, while the horizontal axis corresponds to wavelength in nm. As it is easily seen in the figure, the slope of the transmittance curve 1 is considerably low, with the wavelength range between minimum and maximum transmittance values being rather wide.

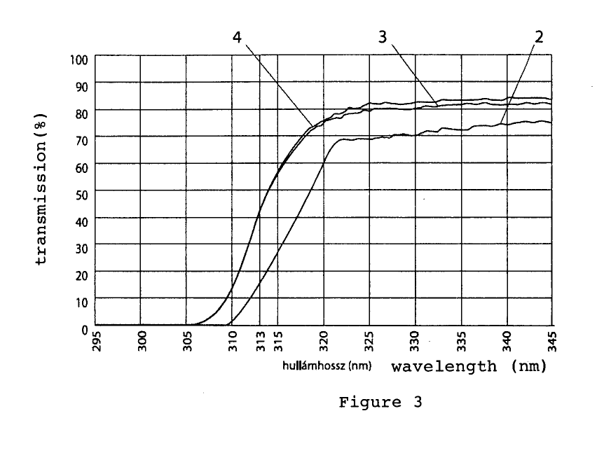

We have conducted experiments with UV filters of different materials and parameters. Filter characteristics are shown in FIG. 3.

Parameters of the transmittance curve designated with the reference numeral 2:

| | |
|---|---|
| material | poly(ethylene terephthalate) PET |
| thickness | 36 μm |

Parameters of the transmittance curve designated with the reference numeral 3:

| material | poly(ethylene terephthalate) PET |
|---|---|
| thickness | 12 μm |
| HAZE factor | 1.5 |

Parameters of the transmittance curve designated with the reference numeral 4:

| material | poly(ethylene terephthalate) PET |
|---|---|
| thickness | 12 μm |
| HAZE factor | 0.5 |

Transmittance curves indicate that at 305 nm transmittance is lower than 5%, the curves reaching a transmittance value of 59% at 320 nm. By selecting the material, the thickness, and the HAZE factor of the filter an arbitrary UVB/UVA ratio (preferably 1:1) may be provided while complying with the prescribed 0.3 W/m² erythema protection value.

The main advantage of the UV filter according to the invention lies in that it can be manufactured easily and cheaply and has a wide range of possible applications. The UV filter may be applied in conventional sunbeds or sunbooths such that the filter is placed on the sunbed's acrylic surface. The filter may also be applied in phototherapy equipment by placing it in front of the light source. The characteristics of the filter allow it to be placed directly on the surface of a low-pressure UV light source.

The invention has been described herein referring to preferred embodiments, but our requested scope of protection is not restricted to the described embodiments. It is obvious to a person skilled in the art that it is possible to modify, improve, and combine the presented exemplary embodiments within the scope of protection specified by the claims.

1. Arrangement for optimally adjusting the UVB to UVA ratio of artificial UV light, particularly for solar cosmetic purposes, comprising at least one light source emitting in both the UVB and UVA wavelength ranges, where the major part of spectral power of the light source falls in the range between 321-400 nm, and where at least one single-layer UV filter is arranged between the light source and the irradiated surface, characterised by that the transmittance of the at least one UV filter at the wavelength of 305 nm or below is lower than 5% and at the wavelength of 320 nm or above is greater than or equal to 59%, with the transmission of the filter at the wavelength of 313 nm being in the range of 12-59%.

2. The arrangement according to claim 1, characterised by that the filter is made from polyester, preferably from poly(ethylene terephthalate) (PET) or poly(ethylene naphthalate) (PEN) or the derivatives thereof.

3. The arrangement according to claim 1, characterised by that the filter is made from a cellulose-based material.

4. The arrangement according to claim 1, characterised by that the material of the filter is polyethylene (PE) coated with indium tin oxide (ITO) coating, or polypropylene (PP), or the copolymers thereof, or acrylic.

5. The arrangement according to claim 4, characterised by that the thickness of the ITO coating is between 0.1-5 μm.

6. The arrangement according to claim 1, characterised by that the thickness of the filter is lower than 100 μm.

7. The arrangement according to claim 1, characterised by that the HAZE factor value of the filter is between 0-5, and preferably between 0-2.

8. The arrangement according to claim 1, characterised by that the filter is at least partially perforated.

9. The arrangement according to claim 8, characterised by that the shape and/or the size of perforations varies across the surface of the filter.

10. The arrangement according to claim 1, characterised by that the light source is a low-pressure UV tanning lamp, and the filter is disposed directly on the surface of the light source.

11. The arrangement according to claim 2, characterised by that the light source is a low-pressure UV tanning lamp, and the filter is disposed directly on the surface of the light source.

12. The arrangement according to claim 3, characterised by that the light source is a low-pressure UV tanning lamp, and the filter is disposed directly on the surface of the light source.

14. The arrangement according to claim 4, characterised by that the light source is a low-pressure UV tanning lamp, and the filter is disposed directly on the surface of the light source.

15. The arrangement according to claim 5, characterised by that the light source is a low-pressure UV tanning lamp, and the filter is disposed directly on the surface of the light source.

16. The arrangement according to claim 6, characterised by that the light source is a low-pressure UV tanning lamp, and the filter is disposed directly on the surface of the light source.

17. The arrangement according to claim 7, characterised by that the light source is a low-pressure UV tanning lamp, and the filter is disposed directly on the surface of the light source.

18. The arrangement according to claim 8, characterised by that the light source is a low-pressure UV tanning lamp, and the filter is disposed directly on the surface of the light source.

19. The arrangement according to claim 9, characterised by that the light source is a low-pressure UV tanning lamp, and the filter is disposed directly on the surface of the light source.
